(19) United States
(12) Patent Application Publication
(10) Pub. No.: US 2004/0023258 A1
(43) Pub. Date: Feb. 5, 2004

(54) Method and system or detecting nucleic acids

(76) Inventors: Fernando Patolsky, Jerusalem (IL); Amir Lichtenstein, Tel Aviv (IL); Itamar Willner, Mevasseret Zion (IL); Yossi Weizmann, Ramat HaGolan (IL)

Correspondence Address: BROWDY AND NEIMARK, P.L.L.C., 624 NINTH STREET, NW, SUITE 300, WASHINGTON, DC, 20001-5303, US

(21) Appl. No.: 10/363,644

(22) PCT Filed: Aug. 29, 2001
(86) PCT No.: PCT/IL01/00808

(30) Foreign Application Priority Data
Sep. 4, 2000 (IL) 138229

(51) Int. Cl. C12Q 1/68

(52) U.S. Cl. 435/6

(57) ABSTRACT

A method for the detection of a target nucleic acid in a sample containing a mixture of nucleic acids. The method comprises (a) providing a solid surface; (b) attaching to the solid surface an oligonucleotide probe complementary to a segment of the target nucleic acid; (c) contacting the surface with the sample, thereby allowing the probe to bind the target nucleic acid; (d) incubating the bound target nucleic acid with the 4 nucleotide types and a replication biocatalyst thereby forming a multi-stranded nucleic acid assembly, wherein at least one of the nucleotide types is bound by a label; and (e) detecting the label on the multi-stranded nucleic acid assembly, thereby detecting the target nucleic acid. Also disclosed are a system for identifying a target nucleic acid sequence in a sample and a kit for use in the method.

### FIELD OF THE INVENTION

This invention relates to method and system for detecting nucleic acids.

### BACKGROUND OF THE INVENTION

The following references are referred to in the specification by number:

1. Ekins, R., Chu, F. W., Trends Biotechnol., 17, 217 (1999).

2. Takenaka, S., Yamashita, K., Takagi, M., Oto, Y, Kondo, H., Anal. Chem. 72, 1334 (2000).

3. Bardea, A., Dagan, A., Ben-Dov, I., Amit, B., Willner, I., Chem. Commun, 839 (1998).

4. Wang, J., Jiang, M., Nielsen, T. W., Getts, R. C., J. Am. Chem. Soc. 120, 8291 (1998).

5. Patolsky, F., Katz, E., Bardea, A., Willner, I., Langmuir, 15, 3703 (1999).

6. Patolsky, F., Lichtenstein, A., Willner, I., J. Am. Chem. Soc., 122, 418 (2000).

7. Patolsky, F., Ranjit, K. T., Lichtenstein, A., Willner, I., Chem. Commun., 1025 (2000).

8. Southern, E. M. TIG, 12, 110 (1996)

9. WO 00/32813

10. U.S. Pat. No. 5,695,926

The detection of the genetic material of pathogens or autosomal recessive diseases is one of the future challenges of medicine and diagnostics. Current methods for sensitive gene detection include polymer chain reaction (PCR) amplification and secondary signal-amplification routes. Major goals in future gene analysis include the parallel detection of a variety of pathogens and their quantitative assay. Inherent limitations of PCR prohibit the application of the method for quantitative and parallel high throughput analyses.

Microarrays of DNA or “DNA chips” have attracted substantial research efforts for the simultaneous analysis of genetic materials (8). In most of these systems the analyte samples are amplified by PCR cycles, and the micro-arrays act as a sensing interface that lacks amplification capabilities (1).

Previous reports addressed the electrochemical (2) or microgravimetric (3) quartz-crystal microbalance detection of DNA. Several recent studies have reported on attempts to amplify the DNA sensing processes: dendritic, hyperbranched oligonucleotides were employed to enhance the binding of DNA to electrodes (4). The biocatalyzed precipitation of an insoluble product on the electronic transducer, that follows the primary hybridization between the analyte DNA and the probe oligonucleotide, has been used to amplify the sensing event (5). Also, labeled liposomes have been employed as micromembrane interfaces that amplify the primary DNA sensing events by their association to the probe-oligonucleotide/DNA-analyte complex generated on the transducer (6). Similarly, dendritic-type amplification of the analysis of a target DNA has been accomplished by the use of oligonucleotide-functionalized Au-nanoparticles (7). Faradaic impedance spectroscopy or frequency changes of the piezoelectric crystal, were used to transduce the different amplified sensing processes.

WO 00/32813 (9) discloses a method for detecting a target oligonucleotide in a sample comprising contacting the sample with a sensor device having a sensing interface carrying oligonucleotides which are complementary to a portion of the target and to which the target hybridizes. The method further provides verification oligonucleotides which are complementary to another portion of the target and which, after binding to the hybridized target oligonucleotides, are detected on the sensing interface.

U.S. Pat. No. 5,695,926 to Cros et al (10) discloses a procedure for detecting single stranded nucleotide sequences in a sample by a sandwich hybridization technique. The procedure uses a passively fixed capture probe of 11-19 nucleotides and a non-radioactively labeled detection probe.

### SUMMARY OF THE INVENTION

It is an object of the present invention to provide a method and system for detecting target nucleic acids in a sample.

The terms “detect” or “detection” in this specification refer collectively to both a qualitative determination and identification of the target nucleic acid in the sample as well as, at times, a quantitative determination of the level of the target nucleic acid in the sample.

The present invention provides a method for monitoring in real time the hybridization of target nucleic acids to specific probes or probes bound to a transducer surface, and to optionally amplify the signal, e.g. by enzymatic means.

In accordance with a first aspect of the invention, there is provided a method for the detection of a target nucleic acid in a sample containing a mixture of nucleic acids comprising:

(a) providing a solid surface;

(b) attaching to the solid surface an oligonucleotide probe complementary to a segment of the target nucleic acid;

(c) contacting the surface with the sample, thereby allowing the probe to bind the target nucleic acid;

(d) incubating the bound target nucleic acid with the 4 nucleotide types and a replication biocatalyst thereby forming a multi-stranded nucleic acid assembly, wherein at least one of the nucleotide types is bound by a label; and

(e) detecting the label on the multi-stranded nucleic acid assembly, thereby detecting the target nucleic acid.

The target nucleic acid may be any DNA or RNA, such as viral or cellular nucleic acids. The method of the invention is intended to enable the qualitative and/or quantitative detection of such a target nucleic acid in a sample.

The sample may be a biological specimen or a fractionation product thereof containing the nucleic acids; a biological specimen treated to free and solubilize nucleic acids; a specimen treated in a manner so as to digest a nucleotide sequence into smaller nucleic acid sequences; a sample of nucleic acids obtained by a PCR process or any other nucleic acid amplification process; etc. Preferably, the target nucleic acid is single stranded, either naturally or derived from a double stranded nucleic acid molecule after appropriate treatment.

The oligonucleotide probe will typically, but not exclusively, comprise a number of nucleotides completing about one helix of the nucleic acid strand, i.e. about twelve nucleotides. A sequence of twelve oligonucleotides ensures, on the one hand, stable hybridization and, on the other hand, a 12-mer oligonucleotide decreases the chance of binding to an incorrect nucleic acid than in the case of a longer sequence. In the case where the sample is a digested specimen of genomic DNA, or a fractionation product thereof comprising the nucleic acids, there is some is probability, which increases with the length of the capturing oligonucleotide, of binding to an incorrect oligonucleotide, namely an oligonucleotide other than the target oligonucleotide. This probability is lower, as aforesaid in the case of a shorter oligonucleotide. On the other hand, the specificity of binding increases with the length of the oligonucleotide with respect to longer target molecules. A sequence of about 12 nucleotides is preferred as it is optimal as far as ensuring binding stability, on the one hand, and reducing incorrect binding on the other hand. The invention is, however, not limited to such a length of the oligonucleotide probe, and the skilled man of the art will know how to adjust the length of the probe to the requirements of the method.

The solid surface may be any surface to which the oligonucleotide probe can bind.

The four types of nucleotides are DATP, dGTP, dTTP, dCTP. The replication biocatalyst may be a polymerase, e.g. DNA polymerase I, Klenow fragment, when the target nucleic acid is DNA, or a reverse transcriptase when the target nucleic acid is RNA.

The label may be any molecule capable of being detected either directly or indirectly by any type of detection means including electronic, optical, etc. Examples of directly-detectable labels include a fluorophore, a dye and a redox-active substance. Examples of indirectly-detectable labels include a first molecule which may be specifically bound by a second molecule with high affinity. The first molecule is termed a “recognition agent”, while the second molecule is termed a “recognition partner” The recognition agent and the recognition partner constitute together a “recognition couple”. A signal amplifying agent may comprise a recognition partner, which in turn binds to the recognition agent. The signal amplifying agent produces a signal which may be detected.

The recognition couple may, for example, be one of the couples selected from the group of biotin-avidin or biotin-streptavidin, receptor-ligand, sugar-lectin, antibody-antigen (the term “antibody” should be understood as referring to a monoclonal or a polyclonal antibody, to a fraction of an antibody comprising the variable, antigen-biotin binding portion, etc.), etc. The recognition agent may be one member of the aforementioned couples, while the recognition partner will then be the corresponding member of the recognition couple.

In a preferred embodiment of the first aspect of the invention, step (e) of the method of the invention comprises the following sub-steps:

(e1) incubating the nucleic acid assembly with a signal amplifying agent which is bound by the recognition agent to form a recognition couple;

(e2) incubating the recognition couple with a substrate of the signal amplifying agent wherein the signal amplifying agent acts on the substrate to produce a detectable product; and

(e3) detecting the product thereby detecting the target nucleic acid.

The signal amplifying agent may be any molecule capable of producing a detectable signal. A preferred example of such a molecule is an enzyme. Non-limiting examples of suitable enzymes include alkaline phosphatase, glucose oxidase and horseradish peroxidase.

The detectable product may be detected by a variety of known techniques. For example, the product may be detected by precipitating on the solid surface, by producing a color, by fluorescing or by producing an electrochemical signal. The amount of the detectable product produced by the signal amplifying agent may be optically or electronically transduced, either in solution or by the solid surface.

Preferably, the solid surface is functionalized so as to enable it to act as a transducer of the signal produced, directly or indirectly, by the label. For example, the surface may comprise a glass or polymer support such as an electrode or piezoelectric crystal. In accordance with one embodiment of the invention, the solid surface comprises an electrochemical probe for electrical/electrochemical measurements, e.g. for Faradaic impedance spectroscopy measurement or amperometric detection of the target nucleic acid. In addition, detection may also be carried out by a number of other electrochemical techniques known per se based on the control of interfacial electron transfer rates between the solid surface and the surrounding medium. For this electrochemical embodiment of the invention, the solid surface is formed on a conductive matrix on which the probe is bound. Such an electrically conducting matrix may, for example, be made or coated by a metal such as gold, platinum, silver or copper.

In accordance with another embodiment of the invention, the solid surface is a quartz crystal microbalance (QCM) probe in which case the detection of a product is based on measurement of changes in resonance frequency of the probe. Microgravimetric QCM techniques are known per se, and are described, for example, in PCT Application No. WO 97/04314.

In accordance with a still further embodiment of the invention, the solid surface is an electrode, the label is a redox-active substance and step 1(e) of the method of the invention comprises the steps of:

(e1) incubating the nucleic acid assembly with a redox-active biocatalyst which is electrically contacted to the electrode by the redox label in the assembly;

(e2) incubating the electrode and biocatalyst in the presence of a biocatalyst substrate, thereby producing a current response of the electrode; and

(e3) detecting the current response of the electrode thereby detecting the target nucleic acid.

The redox-active substance may be, for example, an electro-active organic compound, a transition metal complex or an organic dye. Examples of an electro-active organic compound include a bipyridinium derivative, a quinone and a pyridinium salt. Examples of a transition metal complex include a ferrocyin derivative, a Ry(II)-polypyridine and an Os(II)-polypyridine complex. Examples of an organic dye include a flavin derivative and an acrydine derivative.

The redox-active biocatalyst may be, for example, an enzyme such as an oxidase, reductase or dehydrogenase. Examples of oxidases include glucose oxidase, lactate oxidase and choline oxidase. These enzymes may interact with redox-active substances such as quinone derivatives and with transition metal complexes such as ferrocyin derivatives. Examples of reductases include nitrate reductase, nitrite reductase and glutathione reductase. These enzymes may interact with redox-active substances such as bipyridinium derivative. An example of a dehydrogenase is glucose dehydrogenase.

The current response of the system may be detected by an electrochemical method such as, for example, amperometric measurement, differential pulse voltammetry or chronoamperimetry.

The redox molecule can be detected directly or through its mediation of the oxidation or reduction of a redox-active biocatalyst, such as an enzyme, which is activated towards its bio-electro catalytic transformation. In addition, the target nucleic acid may also be detected by allowing the biocatalyst to act on the biocatalyst substrate to produce a detectable product. The product is then detected as described above. Either one, two or all of these methods of detection may be used.

In a second aspect of the invention, there is provided a system for identifying a target nucleic acid sequence in a sample containing a mixture of nucleic acids comprising:

(a) a biochip comprising a plurality of arrays of functionalized solid surfaces each of which may act as a transducer, each of the surfaces having bound thereto a probe complementary to a different segment of a target nucleic acid sequence, each of the arrays being specific for a different target nucleic acid sequence;

(b) 4 nucleotide types wherein at least one of the nucleotide types is bound by a label, and

(c) a replication biocatalyst.

The label, biocatalyst and other components of the system are as defined above.

In this aspect of the invention, parallel analysis of multiple samples may be carried out on microarrays of functionalized solid surfaces. For example, if it is desired to determine the identity of a infecting virus in a sample, a DNA chip or bio-chip may be used in which one row of solid surfaces will comprise probes complementary to different segments of the genetic material of one type of virus, a second row will comprise probes complementary to a second type of virus, etc. Application of the sample to the biochip and locating the row which produces a signal will enable identification of the infecting virus. A similar detection system may be used to identify genetic mutants and diseases, in tissue typing, gene analysis and forensic applications.

In a third aspect of the invention, there is provided a kit for the detection of a target nucleic acid sequence in a sample containing a mixture of nucleic acids comprising:

(a) a functionalized solid surface which acts as a transducer and having a probe attached thereto;

(b) 4 nucleotide types wherein at least one of the nucleotide types is bound by a label, and

(c) a replication biocatalyst.

Preferably, the kit further comprises the following components:

(d) a recognition couple, wherein one member of the recognition couple is bound to a signal amplifying agent, and

(e) a substrate of the signal amplifying agent.

The label, biocatalyst and other components of the system are as defined above.

### DETAILED DESCRIPTION OF THE DRAWINGS

In order to understand the invention and to see how it may be carried out in practice, embodiments will now be described, by way of non-limiting example only, with reference to the accompanying drawings, in which:

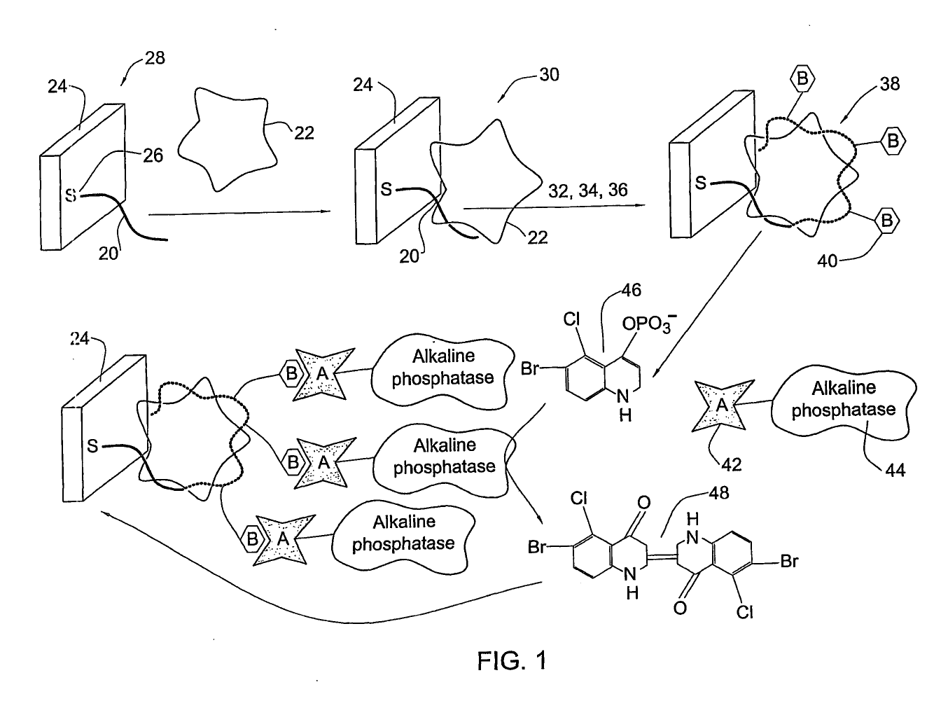

FIG. 1 is a schematic illustration of amplified electronic transduction of the detection of M13φ by the Polymerase-induced replication of DNA and the biocatalyzed precipitation of an insoluble product on the transducer;

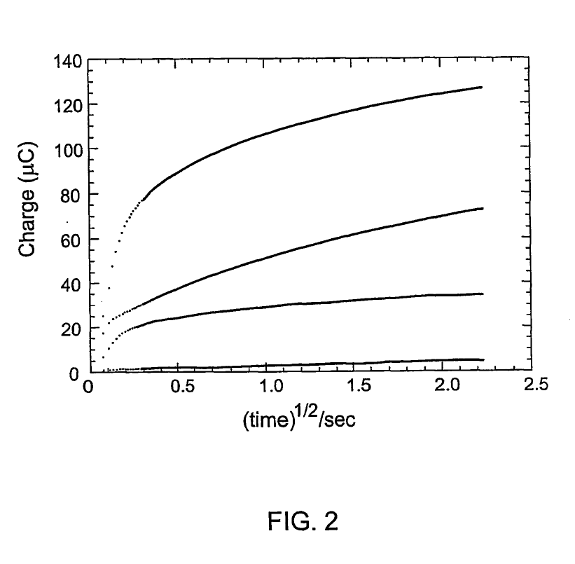

FIG. 2 shows chronocoulometric transients for: (a) A bare Au-electrode; (b) A probe-modified Au-electrode; (c) The probe-modified Au-electrode after hybridization with M13φ, 2.3×10⁻⁹ M, for 1.5 h; (d) The probe-modified electrode after hybridization with M13φ, 2.3×10⁻⁹ M, for 4 h. All transients were recorded in the presence of Ru(NH₃)₆³⁺, 5×10⁻⁵ M, in 10 mM Tris buffer, pH=7.4. The hybridizations were conducted in 0.1 M phosphate buffer, pH=7.5, that included 30% formamide, at room temperature;

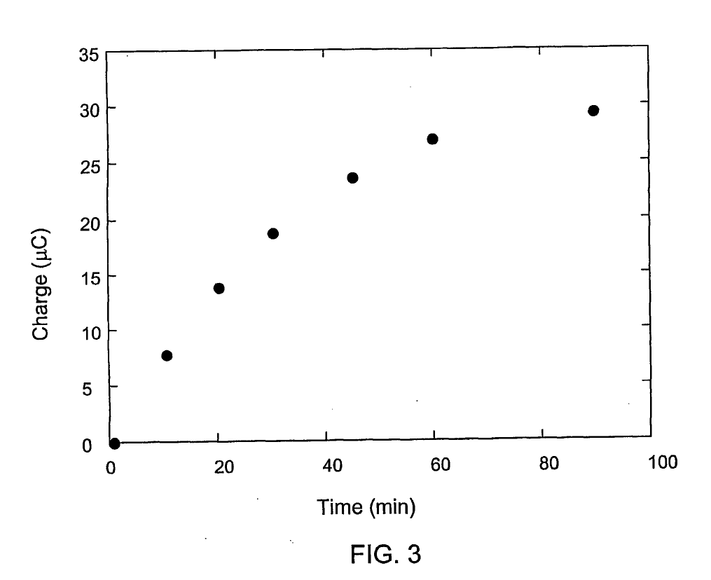

FIG. 3 shows the time-dependent changes of the charge associated with the polymerase-induced polymerization of the double-stranded assembly on the M13φ DNA. The change in the charge is measured by chronocoulometry using Ru(NH₃)₆³⁺ as redox-label in 10 mM Tris buffer, pH=7.4. Polymerization is conducted in the presence of polymerase, 20 U, dGTP; dTTP; dATP; dCTP and biotin-labeled dCTP (1:1:1:2/3:1/3, each base 1 mM, in a solution consisting of 10 mM Tris buffer pH=7.5, 5 mM MgCl₂ and 50 mM KCl. (prior to each chronocoulometric measurement the electrode was washed thoroughly in Tris buffer, 5 mM);

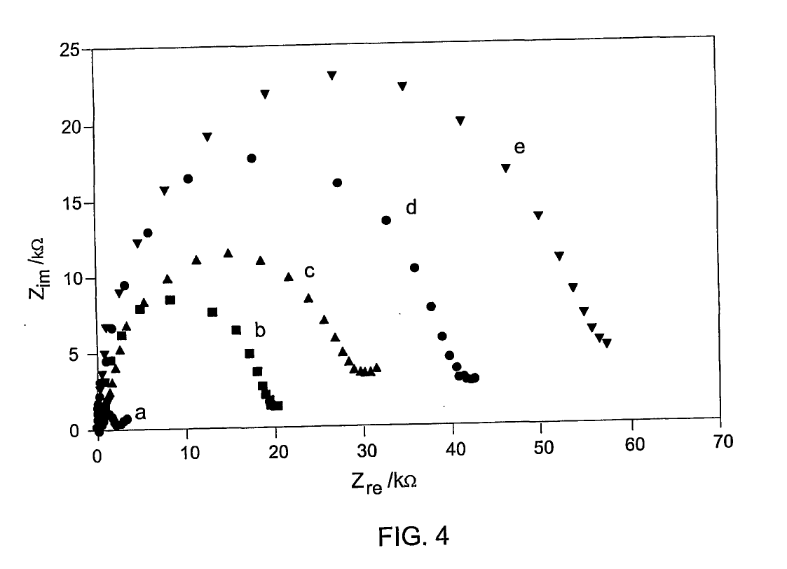

FIG. 4 shows Faradaic impedance spectra (Nyquist plots) of: (a) the probe-functionalized electrode; (b) After hybridization with M13φ, 2.3×10⁻⁹ M; (c) After the polymerase-induced replication and formation of the double-stranded assembly for 45 minutes; (d) After the binding of the avidin-alkaline phosphatase conjugate to the surface; (e) After the biocatalyzed precipitation of the detectable product for 20 minutes in the presence of the substrate, 2*10⁻³ M, in 0.1 M phosphate buffer pH=7.2. The conditions for the hybridization and polymerization are detailed in the captions of FIGS. 2 and 3;

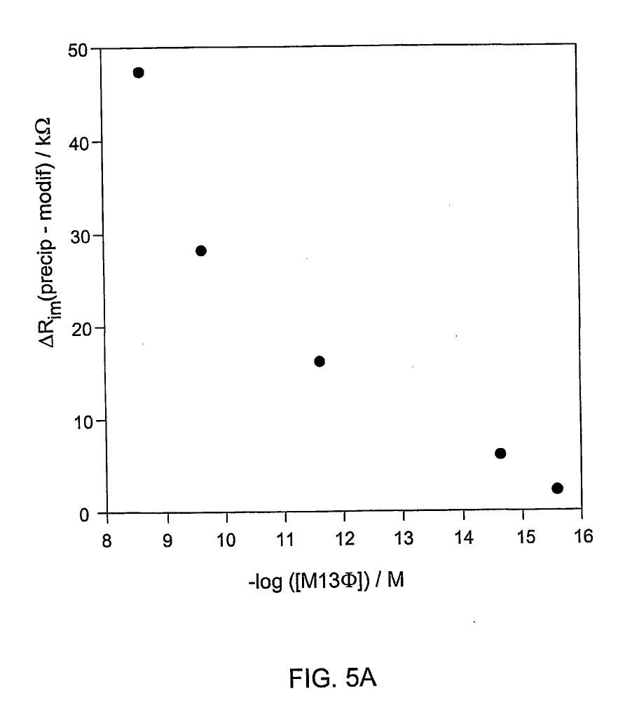

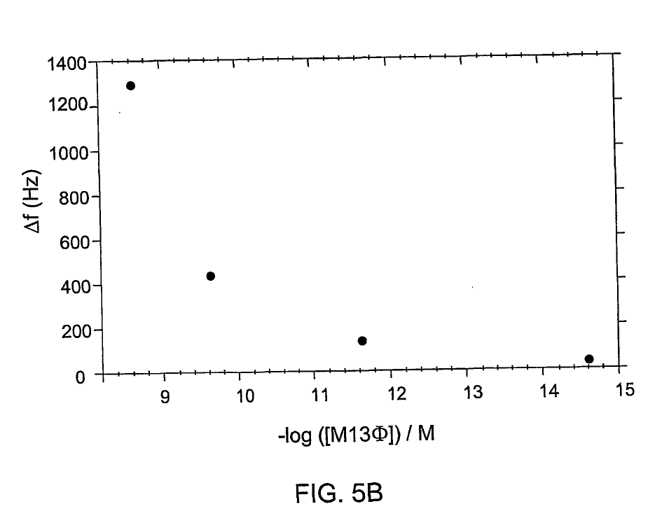

FIG. 5A shows the changes in the electron transfer resistances, ΔRₑₜ, upon the sensing of different concentrations of M13φ by the amplified biocatalyzed precipitation of product on the electrode. The ΔRₑₜ corresponds to the difference in the electron transfer resistance at the electrode after the precipitation of product for 20 minutes and the electrode transfer resistance at the probe-functionalized electrode.

FIG. 5B shows frequency changes of the probe-functionalized Au-quartz crystal upon the sensing of different concentrations of M13φ as a result of the biocatalyzed precipitation of product on the transducer. The conditions for hybridization, polymerization and the biocatalyzed precipitation of product are is detailed in FIG. 4;

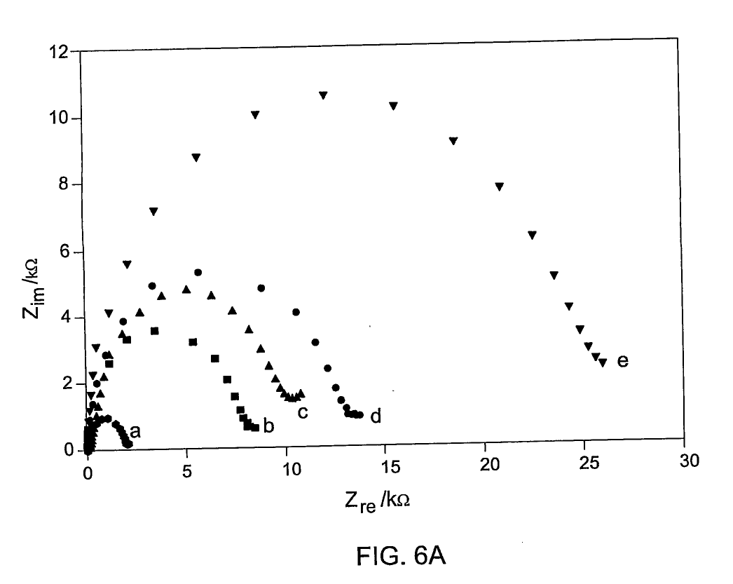

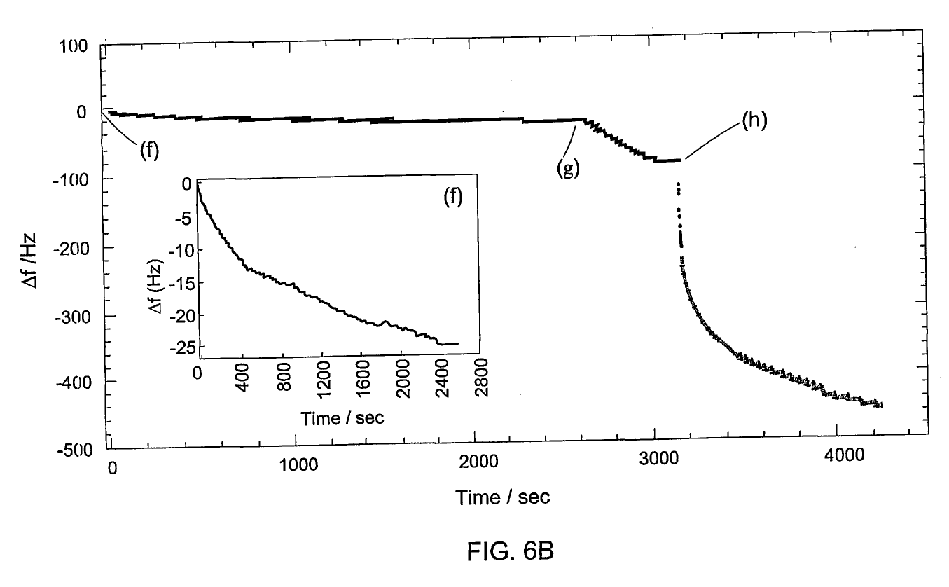

FIG. 6A shows Faradaic impedance spectra (Nyquist plots) upon the amplified sensing of Vesicular Stomatitis Virus RNA: (a) The probe-(5)-functionalized electrode; (b) After hybridization with the VSV RNA, 1×10⁻¹² M, in a solution consisting of 40 mM PIPES, 1 mM EDTA, 400 mM NaCl, 80% formamide, pH=7.5; (c) After the polymerization for 45 minutes in the presence of reverse transcriptase, 80 U, dGTP, dATP, DTP, DTP and biotinylated-dCTP (1:1:2/3:1:1/3 each base 1 mM) in a solution consisting of 50 mM Tris buffer, 40 mM KCl, 8 mM MgCl₂, pH=8.3; (d) After the association of the avidin-alkaline phosphatase conjugate, bulk concentration 1 nM; (e) After the biocatalyzed precipitation of product for 20 minutes in the presence of substrate, 2*10⁻³ in phosphate buffer pH=7.5;

FIG. 6B shows tine-dependent frequency changes of an Au-quartz crystal upon the analysis of the VSV-RNA, 1×10⁻¹¹² M; (f) Frequency changes as a result of the replication of the bound RNA in the presence of reverse transcriptase and dGTP, dATP, dTTP, dCTP and biotinylated-dUTP; (g) Upon the association of the avidin-alkaline phosphatase conjugate; (h) Upon the biocatalyzed precipitation of product. The conditions for the polymerization and biocatalyzed precipitation of product are detailed in FIG. 6A. Inset: Magnification of curve (f);

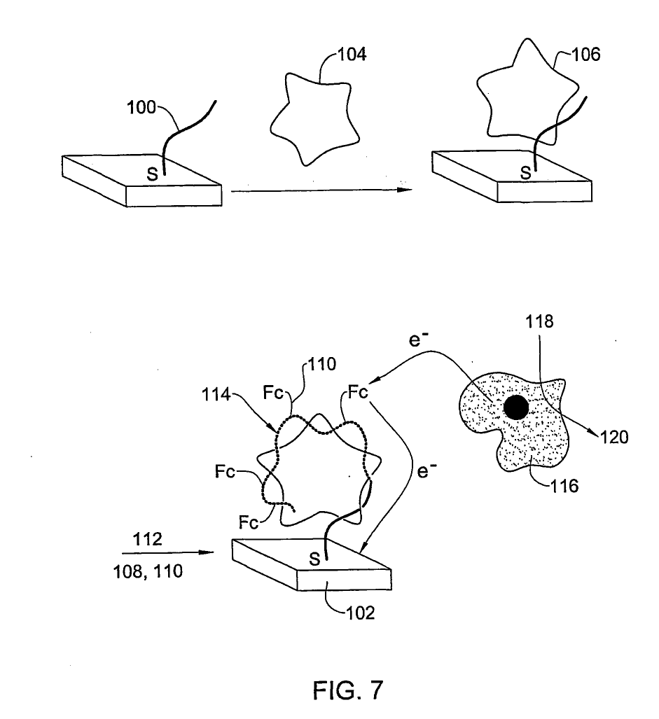

FIG. 7 is a schematic illustration of the generation of the redox-active DNA replicas and the amperometric amplified bioelectrocatalytic analysis of the gene;

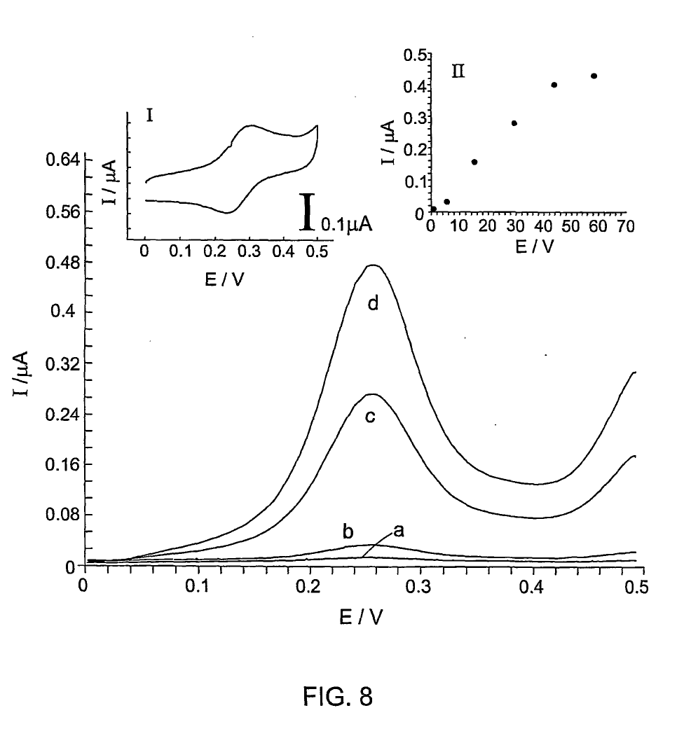

FIG. 8 shows the differential pulse voltammograms (DPV) corresponding to the ferrocene-functionalized replica formed upon the polymerase-induced replication of the double-stranded assembly formed between SEQ. ID. NO: 1 and the M13φ DNA, 1×10⁻⁹ M, at different time intervals of polymerization: (a) Before replication. (b) After 5 minutes. (c) After 30 minutes. (d)After 60 minutes. Insets: (I) The cyclic voltammogram of the ferrocene tethered DNA after 60 minutes of replication, scan-rate 100 mV·s⁻¹. (II) Peak-current of the DPV curves for the ferrocene-tethered DNA at different replication time-intervals. In all experiments the sensing interface was hybridized with M13φ DNA, 1×10⁻⁹ M. Replication was performed in Tris buffer, 20 mM, pH=7.5, that includes 10 mM MgCl₂ and 60 mM KCl, in the presence of polymerase, Klenow fragment, 20 U·mL⁻¹, and dCTP, dATP, dGTP, ferrocene-dUTP, (2), each 1×10⁻³ M;

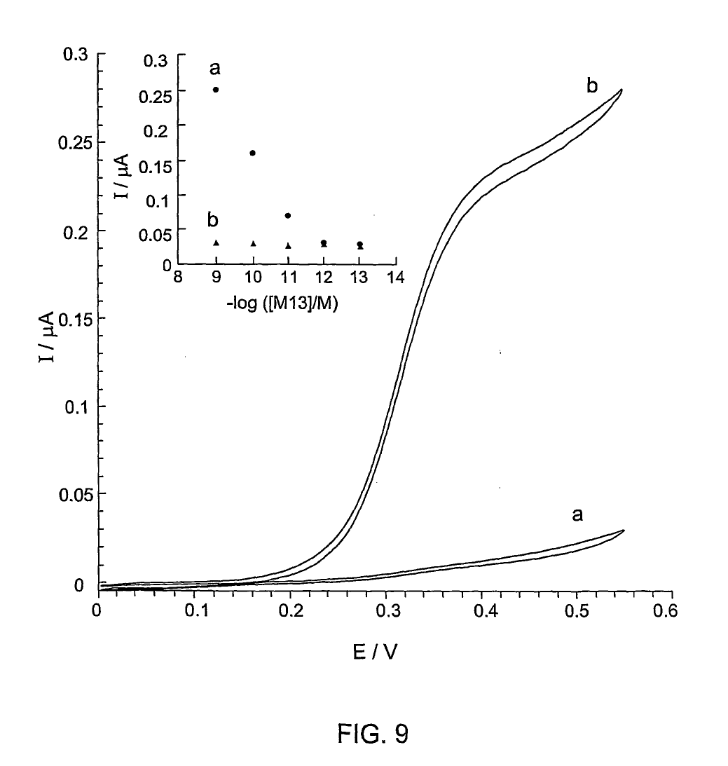

FIG. 9 shows cyclic voltammograms corresponding to: (a) The ferrocene-tethered DNA generated upon analysis of M13φ DNA, 1×10⁻⁹ M, after replication for 60°, in the presence of GOx, 2 mg·mL⁻¹. (b) After addition of glucose, 5c10⁻² M to the system described in (a). Data were recorded in phosphate buffer, 0.1 M, pH=7.4, under argon, scan-rate 2 mV·s⁻¹. Inset: (curve a): Peak-currents of bioelectrocatalytic anodic waves observed upon the analysis of different concentrations of M13φ DNA through the generation of the respective redox-active replica and the mediated electrobiocatalyzed oxidation of glucose by GOx, 2 mg·mL⁻¹, glucose, 5×10⁻² M. (curve b): The peak-currents of the anodic redox-waves upon the analysis of different concentrations of M13φ DNA, through the generation of the respective ferrocene tethered replica, but in the absence of the GOx/glucose amplification system. All cyclic voltammograms were recorded in phosphate buffer, 0.1 M, pH=7.4, under Ar, scan-rate 2 m V·s⁻¹; and

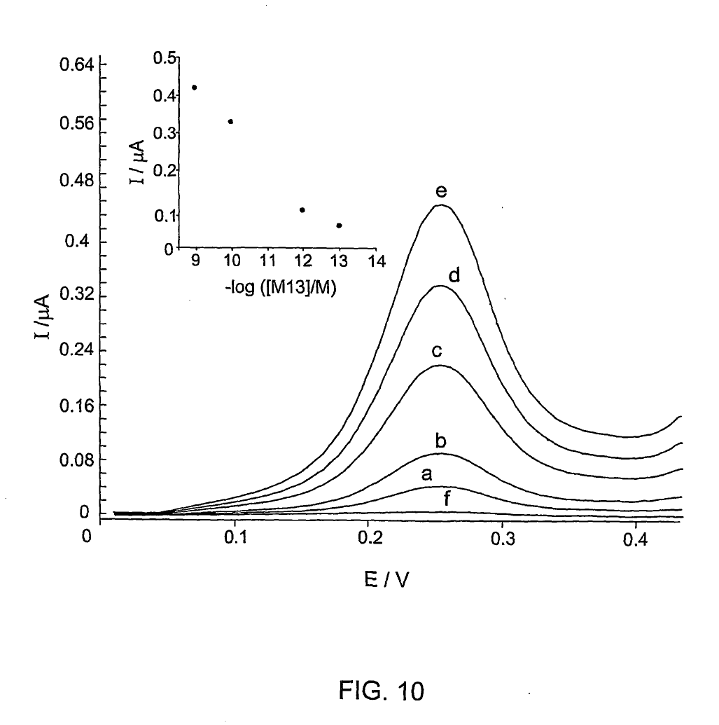

FIG. 10 shows the differential pulse voltammograms (DVP) of the ferrocene-tethered replica formed upon the analysis of different concentrations of M13φ DNA: (a) 1×10⁻¹³ M, (b) 1×10⁻¹² M, (c) 1×10⁻¹¹ M, (d) 1×10⁻¹⁰ M, (e) 1×10⁻⁹ M, (f) a control experiment where denaturated calf thymus DNA, 5×10⁷ M is analyzed according to the scheme in FIG. 7. In all of the experiments a fixed replication time corresponding to 60 minutes was used. Inset: Peak-currents of DPV of the ferrocene-replica formed upon analysis of different concentrations of M13φ DNA.

### DETAILED DESCRIPTION OF THE INVENTION

### EXAMPLE I

One embodiment of the method of the invention is depicted schematically in FIG. 1. The following oligonucleotides were used as probes in the following examples:

(SEQ ID NO: 1)

| | |
|---|---|
| (1) | ′5-HS-(CH₂)₆-CCCCCACGTTGTAAAACGACGGCCAGT-3′ |

(SEQ ID NO: 2)

| | |
|---|---|
| (4) | ′5-HS-(CH₂)₆-CGT TTG ATT ACT GGC CTT GCG GATC-3′ |

(SEQ ID NO: 3)

| | |
|---|---|
| (5) | ′5-HS-(CH₂)₆-ATTTGTAGCCCTTTCGTCCCCTATGTTAGC-3′ |

The sequence (1) is complementary to the cyclic viral gene of mp13 that includes 7229 bases.

A probe thiolated oligonucleotide 20 (e.g. SEQ ID NO: 1), complementary to a segment of a target nucleic acid 22 (e.g. M13 mp8 viral DNA), is assembled on an Au-electrode or an Au-quartz crystal solid surface 24 through a thiol functional group 26. The sensing interface 28 (solid surface+probe) is then reacted with the sample DNA containing the target nucleic acid, and the resulting complex 30 on the transducer is subsequently interacted with dATP, dGTP, dTTP, dCTP 32 and biotinylated-dCTP 34 (ratio 1:1:1:2/31/3, nucleotides concentration of 1 ml in the presence of DNA polymerase I, Klenow fragment 36 (20 U*mL⁻¹). Polymerization and the formation of a double stranded assembly 38 with the target DNA is anticipated to provide the first amplification step of the analysis of the virus-DNA.

A recognition agent in the form of the biotin tags 40 introduced by the polymerase into the double-stranded assembly 38 provides a high number of docking sites for the binding of a conjugate recognition partner-signal amplifying agent in the form of avidin 42-alkaline phosphatase 44. This is followed by the biocatalyzed oxidative hydrolysis of the substrate 5-bromo-4-chloro-3-indolyl phosphate 46 to form the insoluble indigo product 48, that precipitates on the transducer 24, thus providing a second amplification step for the analysis of the target DNA.

Formation of the polymerized double-stranded assembly on the electrode is anticipated to attract a positively-charged redox-label that can be assayed by chronocoulometry (Steele, A. B., Hernlaned, T. M., Tarlov, M., Anal. Chem., 70, 4670 (1998)). This enables continuous monitoring of the polymerization process. The negatively-charged double-stranded assembly is also anticipated to repel a negatively-charged redox-probe, and to enhance the electron-transfer resistance on the transducer surface. The barrier for electron-transfer to a negatively-charged redox-label in solution can be assayed by the Faradaic impedance spectroscopy technique (Millan, K. M., Saraullo, A., Mikkelsen, S. R., Anal. Chem. 66, 2943 (1994)). Furthermore, hybridization, formation of the double-stranded assembly, polymerization and precipitation of the insoluble product alter the mass on the transducer, and hence the entire set of detection steps of the target DNA may be assayed by microgravimetric analysis of the frequency change of a piezoelectric crystal.

The coverage of the probe oligonucleotide on the transducer may be determined by microgravimetric quartz-crystal-microbalance analysis and chronocoulometric experiments, using Ru(NH₃)₆³⁺ as redox-label (Steele, op. cit.). The surface coverage of the probe oligonucleotide on the transducer is controlled by the probe concentration and by the time of incubation of the transducer with the probe solution. Upon interaction of the Au-electrode with the probe SEQ ID NO: 1, 4.2×10⁻⁶ M, for 60 minutes, a surface with optimal surface coverage, corresponding to (6.3±0.3)×10⁻¹¹ mole·cm⁻², for the sensing of M13φ was generated.

FIG. 2 shows the chronocoulometric transients, in the presence of Ru(NH₃)₆³⁺, of the probe SEQ ID NO: l-oligonucleotide-functionalized-electrode, curve (b), and of the sensing interface after hybridization with the analyte DNA for periods of 1.5 and 4 hours, curves (c) and (d), respectively.

After 4 hours of hybridization, the charge associated with the linked redox-probe was estimated to be 54 μC. Assuming that all of the Ru(NH₃)₆³⁺ units linked to the hybridized analyte DNA communicate electrically with the electrode, the surface coverage of the M13 mp8 DNA on the surface is ca. 9.0×10⁻¹³ mole·cm². Thus, only 1.5% of the sensing oligonucleotide units underwent hybridization with the viral-DNA. This value of surface coverage of the hybridized DNA is further supported by microgravimetric quartz-crystal-microbalance, QCM, measurements that reveal a frequency change Δf=−445 Hz upon the binding of the viral DNA to the surface. This frequency change translates to a surface coverage of the hybridized DNA of 1.1×10⁻¹³ mole·cm⁻², that correspond to hybridization to ca. 1.7% of the sensing interface.

FIG. 3 shows the increase in the charge associated with Ru(NH₃)₆³⁺ linked to the double-stranded assembly, as a result of the polymerase-induced formation of the double-stranded assembly with the analyte DNA that acts as template. The charge increases with time, implying that polymerization occurs on the surface, and it levels-off after ca. 60 minutes of polymerization.

It should be noted that the charge associated with the analyte-DNA is 29.2 μC, but the polymerization did not reach the same value. Thus, polymerase-induced polymerization led only to 54% of formation of the double-stranded assembly (on average 3900 bases were polymerized over each analyte-DNA). This might be attributed to steric constraints for the formation of the fully replicated double-stranded assembly on the surface or to the interruption of the Klenow fragment-induced polymerization that is known¹⁸ to occur at specific sites during M13 replication. The partial polymerization on the surface is further reflected by QCM experiments that indicate that polymerization yields a frequency decrease of Δf=−195 Hz, whereas the attachment of the analyte-DNA to the surface results in a frequency change of Δf=440 Hz.

FIG. 4 shows the Faradaic impedance spectra (in the form of Nyquist plots) of the probe SEQ ID NO: 1-oligonucleotide-functionalized electrode, curve (a), after its hybridization with the analyte-DNA, 2.3×10⁻⁹ M, curve (b), the polymerase-induced formation of the double-stranded assembly, curve (c), and after the association of the avidin-alkaline phosphatase conjugate, curve (d), and the subsequent biocatalyzed precipitation of (3) on the surface for 20 minutes, curve (e).

The electron transfer resistance, Rₑₜ, (diameter of the semicircle domains) increases upon the binding of the virus-DNA from 3 kΩ to ca. 20 kΩ. This is consistent with the fact that binding of the high molecular weight DNA electrostatically repels the negatively-charged redox-label, Fe(CN)₆³⁻/Fe(CN)₆⁴⁻ from the electrode surface. The polymerase-induced polymerization and formation of the double-stranded assembly further increases the electron-transfer resistance to ca. Rₑₜ=33 kΩ. It is to be noted that the polymerization does not double the interfacial electron transfer resistance, consistent with the partial polymerization of the double-stranded assembly on the target DNA.

The binding of the conjugate avidin-alualine phosphatase and the subsequent biocatalyzed precipitation of the product (3) on the electrode, results in an insulating layer that introduces a barrier for the interfacial electron transfer, and the electron transfer resistance increases to Rₑₜ=55 kΩ. Similarly, microgravimetric QCM experiments reveal that at this concentration of the analyte-DNA, the biocatalyzed precipitation of (3) on the crystal yields a frequency decrease of Δf=−1300 Hz as a result of the mass-increase on the transducer. The extent of the biocatalyzed precipitation of (3) on the transducers, and consequently the Rₑₜ and Δf transduced signals, are controlled by the concentration of M13 mp8 in the analyte sample, FIGS. 5(A) and 5(B), respectively. It is to be noted that at concentrations of M13 mp8 corresponding to 2.3×10⁻¹⁵ M and 2.3×10⁻¹⁶ M, the hybridization of the analyte DNA with the sensing interface, and the subsequent polymerization, cannot be detected by Faradaic impedance spectroscopy or QCM, due to the low coverage of the hybridized analyte-DNA on the respective transducers. In the electrochemical transduction, a change in the electron transfer resistance of ΔRₑₜ=2.8 kΩ is observed upon the analysis of the DNA, 2.×10⁻¹⁶ M, as a result of the precipitation of (3). In the microgravimetric analysis of the target DNA, 2.3×10⁻¹⁵ M, a frequency change of −36 Hz is observed as a result of the precipitation of (3) on the transducer.

### EXAMPLE II

A series of control experiments was performed to reveal the high specificity of the developed approach for sensing the targeted virus-DNA: The foreign oligonucleotide, SEQ ID NO: 2, that is not complementary to M13 mp8, was assembled on the electrode or Au-quartz crystals, but failed to analyze the target DNA. Also, the SEQ ID NO: 1-functionalized-electrode or QCM crystal was interacted with denatured calf-thymus DNA, 2.3×10⁻⁹ M, and the resulting transducers were subsequently subjected to polymerization, association of avidin-alkaline phosphatase, and the biocatalyzed precipitation of the insoluble indigo product. No hybridization of calf-thymus DNA with the sensing interface could be detected by impedance spectroscopy or QCM measurements.

After the attempt to stimulate the biocatalyzed precipitation of the insoluble product, a frequency change of Δf=−7 Hz was observed, a value that may be considered as the noise level as a result of the non-specific binding of avidin alkaline phosphatase to the sensing interface. Another experiment was carried out where the SEQ ID NO: 1-functionalized electrode or QCM crystal were interacted with denatured calf-thymus DNA, 2.3×10⁻⁹ M, mixed with an extremely low concentration (2.3×10¹⁵ M) of M13φ DNA. The resulting assembly was subjected to polymerization and biocatalyzed precipitation, and a frequency change Δf=−31 Hz, very close to the frequency change value obtained with the target DNA only, was measured.

### EXAMPLE III

A related approach was used for the amplified sensing of the 11161 base RNA of vesicular stomatitis virus (VSV), using reverse transcriptase as the replication biocatalyst The oligonucleotide, SEQ ID NO: 3, was immobilized as the probe sensing interface on an Au-electrode or an Au-quartz crystal, 1.4×10⁻¹¹ mole·cm⁻². FIG. 6(A) shows the Faradaic impedance spectra of the SEQ ID NO: 3-functionalized electrode, curve (a), after the hybridization with the respective 1×10⁻¹² M, RNA, curve (b), after the reverse transcriptase (80 units) replication of the RNA in the presence of DATP, dGTP, dTTP, dCTP and biotinylated-dCTP (ratio 1:1:1:2/3:1/3, base concentration 1 mM), curve (c), after the binding of the avidin-alkaline phosphatase conjugate, curve (d), and upon the biocatalyzed precipitation of the insoluble indigo product on the transducer, curve (e).

Each of these steps increases, as expected, the electron transfer resistance at the electrode surface. For example, the reverse transcriptase replication of the RNA increases the interfacial electron transfer resistance by ΔRₑₜ=4.5 kΩ, and the precipitation of the insoluble product on the electrode increases the electron transfer resistance by ΔRₑₜ=14.0 kΩ. The RNA could be analyzed with a detection limit that corresponds to 1×10¹⁷M. At this concentration, the hybridization process and the biocatalyzed replication of RNA were invisible, yet the alkaline phosphatase precipitation of the insoluble product on the electrode resulted in an amplification route and the interfacial electron transfer resistance increased by ΔRₑₜ=2.2 kΩ. Control experiments revealed that the interaction of the sensing interface with a foreign RNA, 1×10⁻⁹ M, followed by an attempt to stimulate the reverse-tanscriptase polymerization path and the biocatalyzed precipitation of the insoluble product resulted in a minute change in the electron transfer resistance at the electrode, ΔRₑₜ=0.3 kΩ, indicating that the amplified detection of the VSV RNA is selective.

FIG. 6(B) shows the microgravimetric, quartz-crystal-microbalance analysis of the RNA. Hybridization with the VSV RNA, 1×10⁻¹² M results in a frequency decrase of Δf=−72 Hz that indicates a surface coverage of 1% of the sensing interface. The replication of the RNA in the presence of dGTP, dTTP, dCTP and biotinylated-dCTP (1:1:1:2/3:1/3 base concentration 1 mM) in the presence of reverse transcriptase, 80 U, results in a frequency decrease of −26 Hz, curve (f), FIG. 5(B). This frequency decrease translates to an average replication of the surface associated analyte RNA of 36%. Binding of the avidin-alkaline phosphatase conjugate onto the surface is shown in curve (g), Δf=−52 Hz, and the biocatalyzed precipitation of the insoluble indigo product results in a significant decrease in the crystal frequency that corresponds to ca. Δf=400 Hz, curve (h), FIG. 6(B).

### EXAMPLE IV

An embodiment of the method of the invention which forms redox-active DNA replicas that activate bioelectrocatalytic cascades is depicted schematically in FIG. 7. A thiolated 27-base oligonucleotide probe 100 (e.g. SEQ. ID. NO: 1) was assembled on an Au-electrode 102. The surface coverage of the nucleic acid 100 was determined by microgravimetric quartz crystal microbalance experiments (Patolsky, F.; Lichtenstein, A.; Willner, I., J. Am. Chem. Soc. (2001) 123, 5194-5205) to be 2×10⁻¹¹ mole·cm⁻². Subsequently, the monolayer-functionalized electrode was hybridized with different concentrations of the M13φ DNA 104. Hybridization of M13φ DNA with the probe-functionalized electrode was performed for 4 hours in a phosphate buffer solution, 0.1 M, pH=7.5, that included NaCl, 0.2 M. Microgravimetric quartz-crystal-microbalance experiments indicate that in the presence of a bulk concentration of M13φ DNA corresponding to 1×10⁻⁹ M, the surface coverage of the hybridized cyclic DNA is 1.5% of the primer probe coverage.

The double-stranded assembly 106 is interacted with the nucleotide mixture dNTP 108 that includes the synthetic ferrocene-tethered dUTP 110 as redox-labeled nucleotide, in the presence of polymerase, Klenow fragment I 112. The N-hydroxysuccihnimide ester of ferrocenecarboxylic acid was prepared according to Adamczyk, M; Fishpaugh, J. R.; Heuser, k. J., Bioconjugate Chem., (1997) 8, 253-255. The NHS-ferrocene ester, 5 mg, was added to a stirred solution of the amino-dUTP in a mixture of DMF (100 μL) and TEA (10 μL). The reaction was kept at room temperature for 4 hrs and then loaded onto a DEAE-cellulose column (1×25 cm). A linear gradient from 0.05 to 0.35 M TEAB (pH=7.4) was used. Fractions containing the ferrocene-labeled dUTP were collected, partially desalted, and lyophilized. The formula of the synthetic ferrocene-tethered dUTP is as follows:

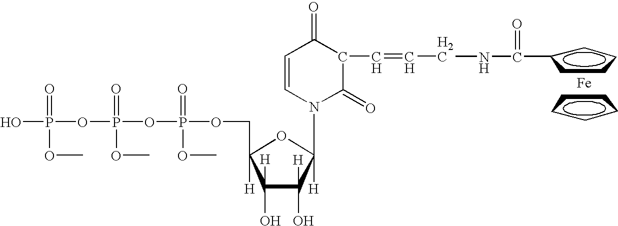

Replication of the target DNA results in a ferrocene-labeled, redox-active, DNA replica 114 that can be analyzed electrochemically. Furthermore, as the ferrocene units act as electron transfer mediators that contact biocatalyst redox-enzymes 116, e.g. glucose oxidase, with electrodes 102, the bioelectrocatalytic oxidation of a biocatalyst substrate 118, e.g. glucose, to a detectable product 120, e.g. gluconic acid, provides an amplification route for the primary generation of the redox-active replica.

FIG. 8 shows the differential pulse voltammograms (DPV) corresponding to the ferrocene-functionalized replica formed upon the polymerase-induced replication of the double-stranded assembly formed between the probe (SEQ. ID. NO: 1) and the M13φ DNA, 1×10⁻⁹ M, at different time intervals of polymerization. As polymerization proceeds, the electrical-response of the redox-replica increases and it tends to reach saturation after ca. 60 minutes. Inset I in FIG. 8 shows the cyclic voltammogram of the replicated redox-active DNA formed after 60 minutes of polymerization. These results clearly indicate that the ferrocene-labeled-dUTP is incorporated in the replicated DNA.

Coulometric analysis of the redox-wave of the ferrocene units after 60 minutes of replication, (shown in inset II of FIG. 8) indicates that ca. 3.6×10¹¹ moles·cm² of ferrocene are electrically-contacted with the electrode. A parallel replication process using an Au-quartz crystal as transducer reveals a frequency change of Δf=−67 Hz upon the polymerization for 60 minutes, indicating a replication efficiency of ca. 59%. Knowing the surface coverage of the redox-label associated with the replicated DNA, it is estimated that the average loading of a DNA replica with the ferrocene units corresponds to ca. 350 ferrocene units per replica (9% of all added bases). It should be noted that at a 59% yield of replication, and taking into account the number of A-bases, only ca. 40% of the ferrocene units that are incorporated in the DNA replica communicate with the electrode. This is probably due to the dimensions of the M13φ DNA that blocks the electrical communication of remote ferrocene units with the electrode.

FIG. 9, curve b, shows the cyclic voltammogram of the ferrocene functionalized replica/M13 DNA double-stranded assembly in the presence of glucose oxidase, GOx, 1 mg·mL⁻¹ and glucose, 100 mM. An electrocatalytic anodic current is observed at the ferrocene-units oxidation potential. Control experiments reveal that no electrocatalytic current is observed in the absence of GOx or glucose. Also, replication of the target M13φ DNA with the nucleotide mixture dNTP/polymerase without the incorporation of ferrocene-tethered dUTP does not yield any electrocatalytic current in the presence of GOx/glucose. Thus, the tethered ferrocene-components mediate the GOx oxidation of glucose.

As the surface coverage of the sensing interface by the M13φ DNA is controlled by its bulk concentration, the electrical response of the replicated ferrocene units, and the electrocatalytic anodic currents resulting in interaction with GOx/glucose are controlled by the bulk concentration of the M13φ DNA. FIG. 10 shows the differential pulse voltammograms of the ferrocene-DNA replicas generated upon the polymerization of the double stranded assemblies formed between the probe-interface and different concentrations of M13φ DNA for a fixed time-interval corresponding to 60 minutes. As the bulk concentration of M13φ increases, the DPV response of the system is enhanced.

Curve (f) of FIG. 10 shows the DPV observed upon an attempt to analyze by the probe-functionalized electrode the foreign denaturated calf thymus DNA followed by an attempt to perform the polymerase-induced replication in the presence of ferrocene-tethered dUTP, dCTP, dATP and dGTP. The lack of any amperometric signal indicates that no non-specific adsorption of ferrocene-tethered dUTP onto the electrode takes place, and that the generation of the redox-response of the ferrocene-tethered replica is a result of the specific formation of a double-stranded assembly between the probe and M13φ DNA.

The resulting redox-tethered, double-stranded interfaces, formed in the presence of different concentrations of the analyte DNA, were then interacted with GOx/glucose as a bioelectrocatalytic amplification system. Curve (a) in the inset of FIG. 9 shows the calibration curve corresponding to the amperometric responses of the ferrocene-tethered DNA replicase in the presence of GOx/glucose as biocatalytic amplification system at different concentrations of M13φ DNA. For comparison, the amperometric responses of the ferrocene-tethered-replicase without GOx and glucose (recorded at a scan-rate of 2 mV·s⁻¹) are presented in curve (b).

1. A method for the detection of a target nucleic acid in a sample containing a mixture of nucleic acids comprising:
(a) providing a solid surface;
(b) attaching to said solid surface an oligonucleotide probe complementary to a segment of said target nucleic acid;
(c) contacting said surface with said sample, thereby allowing said probe to bind said target nucleic acid;
(d) incubating the bound target nucleic acid with the 4 nucleotide types and a replication biocatalyst thereby forming a multi-stranded nucleic acid assembly, wherein at least one of said nucleotide types is bound by a label; and
(e) detecting said label on said multi-stranded nucleic acid assembly, thereby detecting said target nucleic acid.

2. A method according to claim 1 wherein said nucleic acid is single stranded and said multi-stranded nucleic acid assembly is double stranded.

3. A method according to claim 1 wherein said label is selected from the group consisting of a fluorophore, a dye and a redox-active substance.

4. A method according to claim 1 wherin said solid surface is a functionalized solid surface which may act as a transducer.

5. A method according to claim 4 wherein said functionalized solid surface comprises a glass or polymer support.

6. A method according to claim 5 wherein said support is an electrode or a piezoelectric crystal.

7. A method according to any of claims 1-6 wherein said label is a recognition agent.

8. A method according to claim 7 wherein step 1(e) of claim 1 comprises the steps of:
(e1) incubating said nucleic acid assembly with a signal amplifying agent which is bound by said recognition agent to form a recognition couple;
(e2) incubating said recognition couple with a substrate of said signal amplifying agent wherein said signal amplifying agent acts on said substrate to produce a detectable product; and
(e3) detecting said product thereby detecting said target nucleic acid.

9. A method according to either of claims 7 or 8 wherein said recognition couple is selected from the group consisting of biotin-avidin, receptor-ligand, sugar-lecithin and antigen-antibody.

10. A method according to any of claims 7-9 wherein said detectable product is detected by precipitating on said solid surface, by producing a color, by fluorescing or by producing an electrochemical signal.

11. A method according to any of claims 1-10 wherein said replication biocatalyst is a polymerase when the target nucleic acid is DNA or a reverse transcriptase when the target nucleic acid is RNA.

12. A method according to any of claims 7-11 wherein said signal amplifying agent comprises a molecule capable of producing a detectable signal.

13. A method according to claim 12 wherein said molecule is an enzyme.

14. A method according to claim 13 wherein said enzyme is alkaline phosphatase, glucose oxidase or horseradish peroxidase.

15. A method according to claim 14 wherein said substrate is 5-bromo-4-chloro-3-indolyl phosphate.

16. A method according to any of claims 1-15 wherein said target nucleic acid is DNA or RNA.

17. A method according to claim 16 wherein said DNA or RNA is selected from the group consisting of viral DNA, cellular DNA, viral RNA and cellular RNA.

18. A method according to any of claims 1-17 wherein the amount of detectable product produced by the signal amplifying agent is optically or electronically transduced.

19. A method according to claim 18 wherein the amount of detectable product is transduced by said solid surface.

20. A method according to claim 18 wherein the amount of detectable product is transduced in solution.

21. A method according to any of claims 1-19, wherein said functionalized solid surface comprises an electrochemical probe.

22. The method of claim 21, wherein said detection is based on Faradaic impedance spectroscopy or amperometric measurements.

23. A method according to any of claims 1-19, wherein said functionalized solid surface comprises a microbalance quartz-crystal probe.

24. The method of claim 23, wherein said detection is based on a microgravimetric quartz-crystal microbalance (QCM) analysis.

25. A method according to claim 1 wherein said solid surface is an electrode, said label is a redox-active substance and wherein step 1(e) of claim 1 comprises the steps of:
(e1) incubating said nucleic acid assembly with a redox-active biocatalyst which is electrically contacted to the electrode by the redox label in the assembly;
(e2) incubating said electrode and biocatalyst in the presence of a biocatalyst substrate, thereby producing a current response of the electrode; and
(e3) detecting the current response of the electrode thereby detecting said target nucleic acid.

26. A method according to claim 25 wherein said redox-active substance is selected from the group consisting of an electro-active organic compound, a transition metal complex and an organic dye.

27. A method according to claim 26 wherein said electro-active organic compound is selected from the group consisting of a bipyridinium derivative, a quinone and a pyridinium salt.

28. A method according to claim 26 wherein said transition metal complex is selected from the group consisting of a ferrocyin derivative, a Ry(II)-polypyridine and an Os(II)-polypyridine complex.

29. A method according to claim 26 wherein said organic dye is selected from the group consisting of a flavin derivative and an acrydine derivative.

30. A method according to claim 25 wherein said redox-active biocatalyst is an enzyme selected from the group consisting of oxidases, reductases and dehydrogenases.

31. A method according to claim 30 wherein said oxidases are selected from the group consisting of glucose oxidase, lactate oxidase and choline oxidase.

32. A method according to claim 30 wherein said reductases are selected from the group consisting of nitrate reductase, nitrite reductase and glutathione reductase.

33. A method according to claim 30 wherein said dehydrogenase is glucose dehydrogenase.

34. A method according to claim 25 wherein said current response of the electrode is detected by an electrochemical method selected from the group consisting of amperometric measurement, differential pulse voltammetry and chronoamperimetry.

35. A method according to claim 25 wherein in step e2, said biocatalyst acts on said biocatalyst substrate to produce a detectable product, and said product is detected thereby allowing detection of said target nucleic acid.

36. A system for identifing a target nucleic acid sequence in a sample containing a mixture of nucleic acids comprising:
(a) a biochip comprising a plurality of arrays of functionalized solid surfaces each of which may act as a transducer, each of said surfaces having bound thereto a probe complementary to a different segment of a target nucleic acid sequence, each of said arrays being specific for a different target nucleic acid sequence;
(b) 4 nucleotide types wherein at least one of said nucleotide types is bound by a label, and
(c) a replication biocatalyst.

37. A system according to claim 36 wherein said different target nucleic acid sequences are nucleic acid sequences of different pathogenic microorganisms.

38. A system according to claim 36 wherein said different target nucleic acid sequences are nucleic acid sequences related to different genetic diseases.

39. A system according to claim 36 wherein said different target nucleic acid sequences are nucleic acid sequences of different tissues.

40. A system according to claim 36 wherein said different target nucleic acid sequences are nucleic acid sequences of different individuals.

41. A kit for the detection of a target nucleic acid sequence in a sample containing a mixture of nucleic acids comprising:
(a) a functionalized solid surface which acts as a transducer and having a probe attached thereto;
(b) 4 nucleotide types wherein at least one of said nucleotide types is bound by a label, and
(c) a replication biocatalyst.

42. A kit according to claim 41 further comprising:
(d) a recognition couple, wherein one member of said recognition couple is bound to a signal amplifying agent, and
(e) a substrate of said signal amplifying agent.
